(19) United States
(12) Patent Application Publication
(10) Pub. No.: US 2019/0060607 A1
(43) Pub. Date: Feb. 28, 2019

(54) CATHETER TRAY

(71) Applicants: TERUMO CORPORATION, Tokyo (JP); FUJIMORI SANGYO CO., LTD., Tokyo (JP)

(72) Inventors: Hisanobu Yabu, Tokyo (JP); Yuichi Tada, Kanagawa (JP)

(21) Appl. No.: 16/110,142

(22) Filed: Aug. 23, 2018

(30) Foreign Application Priority Data
Aug. 25, 2017 (JP) 2017-161887

(51) Int. Cl. A61M 25/00

(52) U.S. Cl. CPC A61M 25/002

(57) ABSTRACT

To provide a catheter tray that is compact, can reduce costs, can reduce the amount of garbage on the hospital or other catheter user side, and can suppress curling of the catheter.

A proximal portion 90 of a catheter 9 is held by a proximal holding portion 11 of a catheter tray 10. A wound body 93 is obtained by winding a flexible shaft 91 of the catheter 9, and a second portion 93b other than a first portion 93a in a circumferential direction of the wound body 93 is allowed to protrude while holding the first portion 93a by the winding holding portion 20 of the catheter tray 10.

### BACKGROUND OF THE INVENTION

### Field of the Invention

The present invention relates to a catheter tray that holds a catheter, and particularly relates to a catheter tray used for the packaging of a catheter when shipping after manufacturing.

### Description of the Related Art

Catheters used in medical treatments and the like include a flexible, wire-like shaft that elongates from a proximal portion see Patent Literature 1: Japanese Patent No. 4,863,869). The shaft can be inserted into a human body.

Catheters, as products, are stored in catheter trays after manufacturing. The entire catheter tray is then stored in a packing bag and shipped. A user such as a doctor opens the packing bag and removes the catheter from the catheter tray in order to use the catheter. The packing bag and the catheter tray are disposed of as garbage.

Since the shaft of the catheter is long, the catheter is typically set in the catheter tray in a state in which the shaft of the catheter is wound up. The diameter of the wound body of the shaft is, for example, about 10 cm to several tens of cm. Conventional catheter trays are large and, in order to hold the entire wound body, have vertical and horizontal dimensions that are larger than the diameter of the wound body. Consequently, a large amount of material is used, molds are large, and production costs are incurred. Additionally, on the hospital or other user side, the catheter tray is simply thrown away as garbage after the catheter has been removed, and these large catheter trays increase the amount of garbage.

The catheter tray can be made smaller if the winding diameter of the catheter is reduced. However, doing so gives rise to the risk of the catheter becoming curled.

In light of this, an object of the invention is to provide a catheter tray that is compact, can reduce costs, can reduce the amount of garbage, and can suppress curling of the catheter.

### BRIEF SUMMARY OF THE INVENTION

To solve the problem described above, the invention is a catheter tray that holds a catheter including a flexible shaft extending from a proximal portion, the catheter tray including:

a proximal holding portion holding the proximal portion; and

a winding holding portion allowing a second portion, other than a first portion in a circumferential direction of a wound body obtained by winding the shaft, to protrude while holding the first portion.

With the catheter tray, it is sufficient that the winding holding portion is of a size capable of holding only the first portion in the circumferential direction of the wound body of the catheter. As such, the catheter tray can be made compact and cost can be reduced. Moreover, it is not necessary to reduce the winding diameter of the wound body, and curving can be suppressed. Compacting the catheter tray leads to a reduction in the amount of garbage on the hospital or other catheter user side.

It is preferable that a width of the winding holding portion is less than or equal to a radius of the wound body. As a result of this configuration, at least half the circumference of the wound body will protrude, and the catheter tray can be reliably made compact.

It is preferable that a winding holding groove having an arc shape is formed in the winding holding portion, the winding holding groove being capable of accommodating the first portion. As a result of this configuration, the wound body can be securely held.

It is preferable that a center angle of the arc that the winding holding groove forms is less than or equal to 180°. As a result of this configuration, the catheter tray can be reliably made compact.

It is preferable that a holder holding the shaft is attached to the catheter tray, a height of the holder being the same as a height of the catheter tray.

### BRIEF DESCRIPTION OF THE SEVERAL VIEWS OF THE DRAWING

FIG. 1 is a plan view of a catheter tray according to an embodiment of the invention in which a catheter held by the catheter tray is illustrated as a two-dot chain line;

FIG. 2 is an enlarged cross-sectional view taken along line II-II of FIG. 1, and illustrates a winding holding portion of the catheter tray; and

FIG. 3 is a perspective view of the catheter tray.

### DETAILED DESCRIPTION OF THE INVENTION

Next, embodiments of the invention are described while referencing the drawings.

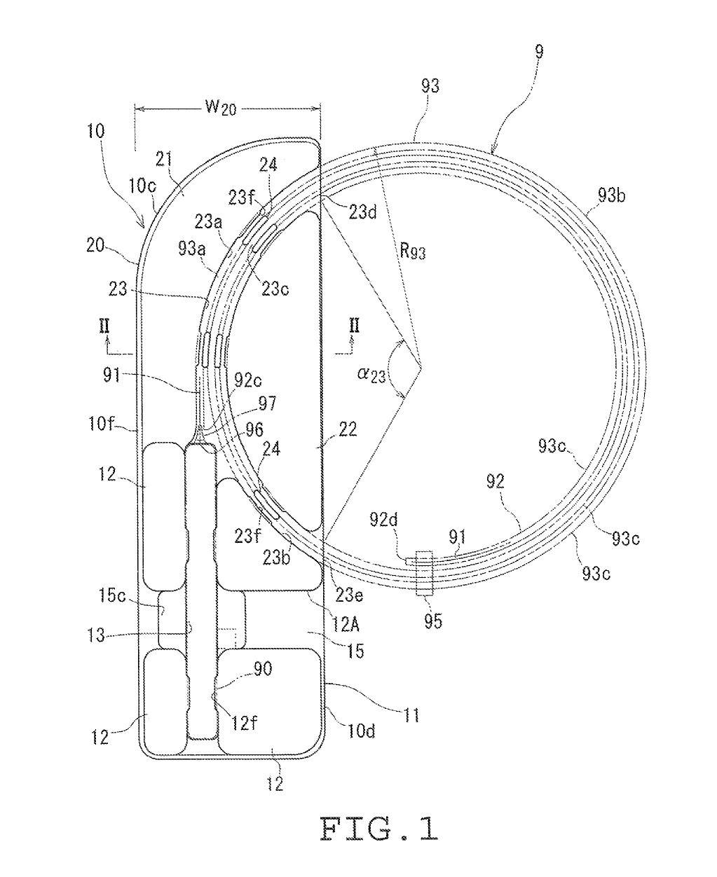

FIGS. 1 to 3 illustrate catheter tray 10. As illustrated in FIG. 1, a manufactured and unused catheter 9 is held by the catheter tray 10. While not illustrated in the drawings, the catheter tray 10 and catheter 9 held therein are stored in a packing bag.

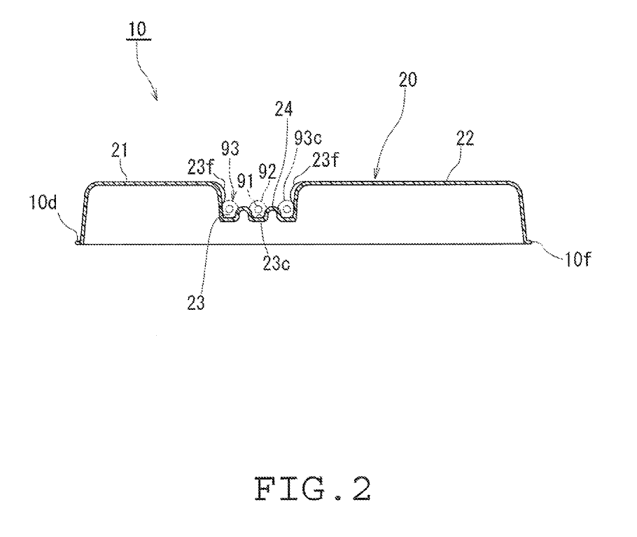

As depicted by the two-dot chain line in FIG. 1, the catheter 9 includes a proximal portion 90 and a shaft 91. The proximal portion 90 is, for example, a straight rod or stick shape, and can be operated by a hand. The proximal portion 90 may be simply a hub without any operation function. The shaft 91 is flexible and extends long from the proximal portion 90. The shaft 91 can be inserted into a human body. The shaft 91 and the proximal portion 90 are coupled at a joint portion 96. The joint portion 96 is protected by a joint protector 97. The joint protector 97 is an anti-kink protector. The joint protector 97 is made from resin and has a function to prevent kink between the proximal portion 90 and the shaft 91, which have different rigidities. As illustrated in FIGS. 1 and 2, an outer periphery of the shaft 91 is covered by a protective tube 92 that is made from resin. As illustrated in FIG. 1, the protective tube 92 and the shaft 91 are wound to form a wound body 93. A distal portion of the joint protector 97 mates with a base end opening portion 92c of the protective tube 92. When in use, the joint protector 97 is removed from the base end opening portion 92c of the protective tube 92. The base end opening portion 92c of the protective tube 92, the joint portion 96, and the proximal portion 90 are disposed in the catheter tray 10 on substantially a straight line. The portion of the catheter tray 10 itself where the base end opening portion 92c is disposed, the portion where the joint portion 96 is disposed, and the portion where the proximal portion 90 is disposed are disposed on substantially a straight line. As a result, breakage of the shaft 91 is prevented.

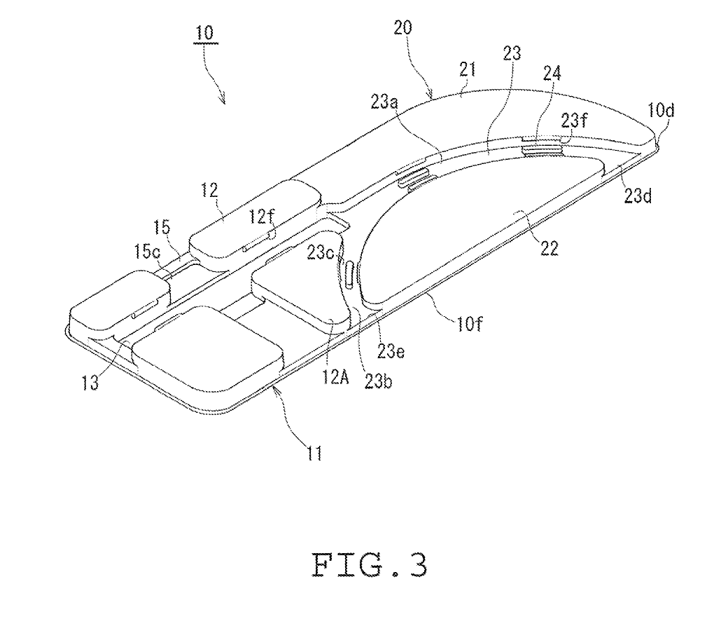

As illustrated in FIGS. 1 and 3, the catheter tray 10 includes a proximal holding portion 11 holding the proximal portion 90, and a winding holding portion 20 holding the wound body and has a vertically long shape.

The material of the catheter tray 10 is a resin such as polyethylene terephthalate (PET). Note that the material of the catheter tray is not necessarily limited to PET, and other resins such as polyethylene (PE) and polypropylene (PP) may be used.

The catheter tray 10 is manufactured by vacuum molding, for example. Note that the manufacturing method of the catheter tray is not necessarily limited to vacuum molding, and other manufacturing methods such as injection molding may be applied.

A width of the catheter tray 1 s about 5 cm to 15 cm. This is a size that is easy to grip in the palm of a hand.

The proximal holding portion 11 is disposed on a first side (the lower side in FIG. 1) in the longitudinal direction of the catheter tray 10. In one example, four (a plurality of) rectangular or triangular polygonal raised portions 12 are provided juxtaposed vertically and horizontally in the proximal holding portion 11. A handle holding groove 13 and a transverse recess 15 are formed by these polygonal raised portions 12. The handle holding groove 13 extends in a linear manner along the longitudinal direction (top to bottom in FIG. 1) of the catheter tray 10.

As illustrated in FIG. 3, a small locking protrusion 12f is formed on an upper side of a wall of each of the polygonal raised portions 12 that faces the handle holding groove 13.

The transverse recess 15 extends in the width direction (left to right in FIG. 1) of the catheter tray 10. The transverse recess 15 traverses a middle portion in the longitudinal direction of the handle holding groove 13. A dimple 15c is formed in the transverse recess 15 at an intersection with the handle holding groove 13. The ends of the transverse recess 15 respectively reach the left and right edges of the catheter tray 10.

As depicted by two-dot chain line in FIG. 1, the proximal portion 90 is stored in the handle holding groove 13. Additionally, the locking protrusion 12f locks edges of a front side surface of the proximal portion 90 (in FIG. 1, the surface of the front side when viewed on paper). As a result, the proximal portion 90 can be securely held.

As illustrated in FIGS. 1 and 3, the winding holding portion 20 is disposed on a second side (the upper side in FIG. 1) in the longitudinal direction of the catheter tray 10. An outer raised portion 21 and an inner raised portion 22 are provided in the winding holding portion 20 and, also, a winding holding groove 23 is formed.

When viewed planarly, the outer raised portion 21 has a substantially arc-like shape. An arc-like corner 10c of the catheter tray 10 is formed by an outer peripheral edge of the outer raised portion 21. An arc-like hypotenuse portion of a triangular raised portion 12A, among the four polygonal raised portions 12 in the proximal holding portion 11, is disposed on an extension of a circumference along an inner peripheral edge of the outer raised portion 21.

The inner raised portion 22 has a partial disk shape.

As illustrated in FIG. 3, heights of the outer raised portion 21 and the inner raised portion 22 are equivalent to each other, and are lower than the polygonal raised portions 12.

Note that the heights of the outer raised portion 21 and the inner raised portion 22 may be the same as the height of the polygonal raised portions 12.

Additionally, a flange 10f is provided on an outer peripheral edge of the catheter tray 10, and a step 10d is formed between the various raised portions 12, 21, and 22 and the flange 10f. However, the flange 10f may be omitted and the various raised portions 12, 21, and 22 may reach to the outer peripheral edge of the catheter tray 10 without the step 10d.

The outer peripheral edge of the catheter tray 10 is step-less and flush along the entire periphery. Accordingly, when the catheter tray 10 is stored in a packing box, the catheter tray 10 is restrained and stabilized in the packing box by the polygonal raised portions 12 and the entire periphery of the outer peripheral edge.

The winding holding groove 23 is formed between the outer raised portion 21 and the triangular raised portion 12A, and the inner raised portion 22. When viewed planarly, the winding holding groove 23 extends in an arc-like manner. An edge on the outer side of the winding holding groove 23 is defined by the outer raised portion 21 and the triangular raised portion 12A. An edge on the inner side of the winding holding groove 23 is defined by the inner raised portion 22.

A center angle α₂₃ of the arc formed by the winding holding groove 23 is preferably less than or equal to 180°, and more preferably, α₂₃=90° to 150°, and yet more preferably, α₂₃ is about 120°.

A width W₂₀ of the winding holding portion 20 and, in turn, a width dimension of the catheter tray 10, is preferably less than or equal to a radius R₉₃ of the wound body 93 of the catheter 9 (W₂₀≤R₉₃).

The handle holding groove 13 and the winding holding groove 23 each have a bottom surface, and the bottom surface of the handle holding groove 13 has a height that differs from a height of the bottom surface of the winding holding groove 23. A transition portion may be formed between the bottom surface of the handle holding groove 13 and the bottom surface of the winding holding groove 23. The joint protector 97 may be disposed on the transition portion.

The handle holding groove 13 merges with an intermediate portion of the winding holding groove 23. The winding holding groove 23 is divided by the merging portion into a groove section 23a between the raised portions 21 and 22 and a groove section 23b between the raised portions 12A and 22. The groove section 23a is longer than the groove section 23b and also has a wider groove width.

The handle holding groove 13 is disposed on a tangent line to the groove section 23a at the merging portion.

Ends of the groove sections 23a and 23b on a side opposite the merging portion, that is, both ends 23d and 23e of the winding holding groove 23 reach the edge of the same side (the right side in FIG. 1) of the catheter tray 10.

As illustrated in FIGS. 1 and 2, a plurality of alignment protrusions 24 is formed on a groove bottom of the winding holding groove 23. Each of the alignment protrusions 24 is formed with a semicircular cross-section, and the longitudinal direction thereof faces the circumferential direction of the winding holding groove 23. It is preferable that each of the alignment protrusions 24 is provided with a curvature, along the longitudinal direction, that matches the winding holding groove 23.

As illustrated in FIGS. 1 and 2, a pair of alignment protrusions 24, 24 is disposed in parallel side-by-side with a gap in the groove width direction (left to right in FIG. 2) in the groove section 23a between the raised portions 21 and 22. Additionally, two pairs (a plurality of pairs) of the alignment protrusions 24, 24 are disposed with a gap in the circumferential direction of the groove section 23a.

As illustrated in FIGS. 1 and 3, one alignment protrusion 24 is disposed in the groove section 23b between the raised portions 12A and 22.

Note that, the number and arrangement of the alignment protrusions 24 are not limited to the aforementioned example and can be changed as appropriate.

As illustrated in FIGS. 1 and 2, a clamping gap 23c is formed between the various alignment protrusions 24 and the raised portions 21, 22, and 12A, and between the pairs of alignment protrusions 24, 24. A width of the clamping gap 23c is less than or equal to the diameter of the protective tube 92.

A locking protrusion 23f is formed on both walls of the winding holding groove 23, at positions corresponding to each of the alignment protrusions 24. The locking protrusion 23f is formed with a semicircular cross-section and extends in the circumferential direction of the winding holding groove 23.

As depicted by the two-dot chain line of FIG. 1, a second portion 93b other than first portion 93a in the circumferential direction of the wound body 93 is made to protrude from the winding holding portion 20, and the first portion 93a is held by the winding holding portion 20.

The first portion 93a is stored in the winding holding groove 23. A boundary between the first portion 93a and the second portion 93b of the wound body 93 passes through both ends 23d and 23e of the winding holding groove 23.

A circumferential length of the first portion 93a is less than or equal to a circumferential length of the second portion 93b and, preferably, is less than the circumferential length of the second portion 93b. A center angle of the first portion 93a corresponds to the center angle α₂₃ of the winding holding groove 23, and is less than or equal to 180°. Accordingly, at least half the circumference of the wound body 93 is the second portion 93b.

Note that, the wound body 93 is wound on the inner peripheral side each wind, from the proximal portion 90 side toward the tip side of the shaft 91. Each wind of the wound body 93 is referred to as a “wound portion 93c.”

As illustrated in FIGS. 1 and 2, each of the wound portions 93c in the first portion 93a is elastically fitted into the clamping gap 23c, and the wound portions 93c are locked with the locking protrusions 23f. Therefore, the wound body 93 can be securely held. As a result, the wound body 93 can be prevented from falling out of the catheter tray 10, even when subjected to a degree of vibration. Furthermore, due to the alignment protrusions 24, the plurality of wound portions 93c in the first portion 93a can be aligned in the radial direction of the wound body 93.

The second portion 93b of the wound body 93 is held by a holder 95 at one or a plurality of locations in the circumferential direction.

At least one holder 95 is disposed at the portion where the protective tube 92 is protruding. For example, the holder 95 is disposed near the winding holding groove end 23e of the catheter tray 10. Additionally, in cases in which a plurality of holders 95 is disposed, a height of the holders 95 may be the same as that of the catheter tray 10. As a result, a positional relationship with the packing box (not illustrated in the drawings) can be fixed. The height of the holder 95 is preferably substantially equivalent to the height of the winding holding groove 23 of the catheter tray 10 or a thickness of the catheter tray 10.

The base end opening portion 92e of the protective tube 92 is positioned in the handle holding groove 13 or in the winding holding groove 23. Additionally, the base end opening portion 92c of the protective tube 92 is preferably open in a trumpet shape to facilitate insertion of the catheter 9, and the catheter tray 10 is preferably molded to match the shape of the base end opening portion 92c.

A tip end opening portion 92d of the protective tube 92 maybe disposed inside the catheter tray 10, and a groove shape matching the shape of the tip end opening portion 92d may be formed in the catheter tray 10.

With the catheter tray 10, it is sufficient that the winding holding portion 20 is of a size capable of holding only the first portion 93a in the circumferential direction of the wound body 93. Accordingly, the catheter tray 10 can be made more compact than when holding the entire wound body 93, and the weight thereof can be reduced. Furthermore, the amount of resin, used as the material of the catheter tray 10, that is needed can be reduced. Moreover, manufacturing molds can be made smaller. As a result, the manufacturing costs of the catheter tray 10 can be reduced.

Moreover, it is not necessary to reduce the winding diameter of the wound body 93 and, as such, curving can be suppressed.

The catheter tray 10 can be manufactured by vacuum molding a resin sheet and, therefore, can be made less expensive than manufacturing by injection molding.

Even though the shape and structure of the catheter tray 10 are simple, the catheter tray 10 can securely hold the catheter 9.

After manufacturing, the catheter 9, as a product, is held in the catheter tray 10 and, in this state, is inserted into a packing box or packing bag (not illustrated in the drawings) together with the catheter tray 10 and shipped.

A user such as a doctor opens the packing box or packing bag and removes the catheter 9 from the catheter tray 10 in order to use the catheter 9. At this time, the holder 95 is removed first. Then, a finger is inserted into the transverse recess 15 and the intermediate portion of the proximal portion 90 is pinched and lifted. As a result, the proximal portion 90 can be easily removed from the proximal holding portion 11. Then, the proximal portion 90 is pulled in the opposite direction of the winding holding portion 20 with respect to the catheter tray 10 (downward in FIG. 1). Thus, the shaft 91 is pulled out of the protective tube 92. As a result, the catheter 9 is separated from the catheter tray 10 and the protective tube 92 and can be used.

The protective tube 92 has higher rigidity than the catheter tray 10. As such, the catheter tray 10 deforms without deforming the protective tube 92 and, as a result, the catheter 9 can be easily and safely removed in a manner less likely to break.

The packing bag, the catheter tray 10, and the protective tube 92 are disposed of as garbage. The amount of garbage can be reduced due to the catheter tray 10 being made smaller.

The invention is not limited to the embodiments described above and various modifications can be made without departing from the scope of the invention.

For example, it is sufficient that the winding holding portion 20 allows the second portion 93b other than the first portion 93a in the circumferential direction of the wound body 93 to protrude, and holds the first portion 93a. Accordingly, the width W₂₀ of the winding holding portion 20 may be greater than or equal to the radius of the wound body 93, and the center angle α₂₃ of the arc formed by the winding holding groove 23 may be greater than or equal to 180°. Additionally, the first portion 93a may be half the circumference of the wound body 93 or more, and the second portion 93b may be half the circumference of the wound body 93 or less.

### INDUSTRIAL APPLICABILITY

The invention can be applied to, for example, the packaging of a catheter to be used in a medical treatment or the like.

1. A catheter tray holding a catheter comprising a flexible shaft extending from a proximal portion, the catheter tray comprising:
a proximal holding port on holding the proximal portion; and
a winding holding portion allowing a second portion, other than a first portion in a circumferential direction of a wound body obtained by winding the shaft, to protrude while holding the first portion.

2. The catheter tray according to claim 1, wherein a width of the winding holding portion is less than or equal to a radius of the wound body.

3. The catheter tray according to claim 1, wherein a winding holding groove having an arc shape is formed in the winding holding portion, the winding holding groove being capable of accommodating the first portion.

4. The catheter tray according to claim 3, wherein a center angle of the arc formed by the winding holding groove is less than or equal to 180°.

5. The catheter tray according to claim 1, wherein a holder holding the shaft is attached to the catheter tray, a height of the holder being the same as a height of the catheter tray.
